(19) United States
(12) Patent Application Publication
(10) Pub. No.: US 2022/0057382 A1
(43) Pub. Date: Feb. 24, 2022

(54) METHOD FOR ESTIMATING THE EFFECTIVENESS OF A TREATMENT BY AN ANTI-TNF ALPHA AGENT IN A PATIENT SUFFERING FROM RHEUMATOID ARTHRITIS AND HAVING AN INADEQUATE RESPONSE TO AT LEAST ONE BIOTHERAPY

(71) Applicant: SINNOVIAL, Seyssins (FR)

(72) Inventors: Philippe GAUDIN, Meylan (FR); Athan BAILLET, Jarrie (FR); Anaïs COURTIER, Voiron (FR); Minh-Vu-Chuong NGUYEN, Le Versoud (FR); Lisa GUIGUE, Haute-Jarrie (FR); Jacques-Eric GOTTENBERG, Strasbourg (FR)

(73) Assignee: SINNOVIAL, Seyssins (FR)

(21) Appl. No.: 17/415,266

(22) PCT Filed: Dec. 18, 2019
(86) PCT No.: PCT/FR2019/053166

§ 371 Date: Jun. 17, 2021

(30) Foreign Application Priority Data
Dec. 19, 2018 (FR) 1873333

(51) Int. Cl. G01N 33/50; G01N 33/564; G01N 33/68

(52) U.S. Cl. CPC G01N 33/5008; G01N 33/564; G01N 2800/52; G01N 2800/102; G01N 33/6893

(57) ABSTRACT

The present invention relates to a method for estimating the effectiveness of treatment with an anti-TNFα agent in a patient with rheumatoid arthritis and who has had an inadequate response to at least one prior biotherapy, consisting in analysing a biological sample of said patient for the expression of a set of biomarkers, the results of which make it possible to determine whether said agent is a treatment that will engender a beneficial response for said patient. The present invention also relates to a system for estimating the effectiveness of said treatment in said patient comprising means for measuring or receiving data, the expression level of said biomarkers and means for processing these data configured to estimate said effectiveness of the treatment in said patient.

### FIELD OF THE INVENTION

The present invention relates to the treatment of rheumatoid arthritis. It more particularly relates to a method for estimating the effectiveness of treatment with an anti-TNFα agent, in particular with Adalimumab (ADA), in a patient with rheumatoid arthritis (RA) and having had an inadequate response to one or more prior biotherapy treatment(s), consisting in analysing a biological sample of said patient vis-à-vis the expression of a set of biomarkers, the correlation of the results obtained for this set of biomarkers making it possible, notably by comparison with reference values, to determine if the anti-TNFα agent is a promising treatment making it possible to lead to a beneficial response for said patient.

The present invention also relates to a system for estimating the effectiveness of said treatment in said patient comprising means for measuring or receiving data of the expression level of said biomarkers and means for processing these data configured to estimate said effectiveness of said treatment in said patient.

### BACKGROUND OF THE INVENTION

Rheumatoid arthritis (RA) is a chronic inflammatory disease characterised by synovitis, joint injuries, functional handicap and a significant increase in mortality.

Early intervention using sDMARDs (synthetic Disease-Modifying Anti-Rheumatic Drugs) is now recognised as being essential for preventing structural joint damage and progressive loss of function. For patients that do not respond to treatment with sDMARDs or who develop an inadequate response to these drugs over time, bDMARDs (biological DMARDs) are one effective additional treatment option (Smolen et al., 2017). In clinical practice, the first choice of biological therapy is normally a TNFα (Tumour Necrosis Factor alpha) inhibitor. Response to treatment can vary in a very significant manner from one patient to another. Around 30% to 40% of patients who start treatment with a TNFα inhibitor develop thereafter an insufficient or inadequate response to these drugs (Souto et al., 2016). The options for the continuation of treatment in TNFα insufficient patients comprise the use of a second biological agent. Given the number of bDMARD treatment options available for clinicians and their effectiveness in the treatment of RA, the passage between different bDMARDs is common practice. That is why, at the present time, the practitioner recommends, for most diseases, a first-line treatment then, in the event of insufficient or inadequate response, a second-line treatment, and so on. However, within this overall strategy, there exists a debate on the effectiveness relating to the use of another TNFα inhibitor (cycle) or a biological agent with a different mode of action (switching). In addition, the probability for the patient having already received an anti-TNFα of responding to another biological treatment decreases progressively as a function of the increasing number of failures of prior treatments (Rendas-Baum et al., 2011). Thus, data from the literature indicate that any early intervention makes it possible to better contain the progression of the disease.

Indeed, the time lost bringing to light a potential therapeutic failure is to the detriment of the effectiveness of the therapeutic action and the well-being of the patient, which in certain cases may have ended up in new symptoms or prejudicial and irreversible consequences in terms of general condition. In addition, they may be costly treatments which may be cumbersome to put in place, which is entirely unsatisfactory when a therapeutic failure is noted.

Considerable progress has been made over recent years concerning the diagnosis, care, treatment and follow-up of patients with chronic inflammatory diseases.

In terms of treatments of chronic inflammatory diseases, and notably chronic inflammatory rheumatisms, biotherapies notably exist which consist in biological molecules, such as proteins, antibodies, having a therapeutic action. Some of them are already used and others are under development.

Among chronic inflammatory diseases, rheumatoid arthritis is an auto-immune disorder of the synovia which is characterised by the proliferation of synoviocytes and the infiltration of inflammatory cells into the joint. Various cytokines play an important role in the regulation of inflammatory diseases.

bDMARDs targeting the tumour necrosis factor (TNF) or the co-stimulation of T-lymphocyte cells for example have enabled considerable progress for the treatment of RA. At present, 9 bDMARDs including the T-lymphocyte cell co-stimulation modulator Abatacept (ABA), anti-IL-6 Tocilizumab (TCZ), anti-CD20 Rituximab (RTX), anti-Interleukine-1 (IL-1) Anakinra (ANK) and anti-TNFα Adalimumab (ADA), Etanercept (ETN), Infliximab (IFX), Golimumab (GOL) and Certolizumab Pegol (CTP), are approved for the treatment of rheumatoid arthritis. However, the responses to each biological agent vary for each individual. Consequently, making an optimal choice of one or more bDMARD(s) within a therapeutic window of opportunity is essential to obtain effectiveness of treatment, which proves to be very expensive. Indeed, the chances of success of a biological treatment dwindle as a function of the increasing number of therapeutic failures with biotherapy (Rendas-Baum et al., 2011).

The practitioner lacks however elements at his disposal to help him in his therapeutic choice. A tool capable of providing the clinician with a score of probability of response or non-response to a treatment would certainly be welcome.

In particular, there lacks at present very early biomarkers which can, among other things, provide guidance with regard to the possible response or non-response to a biological or conventional background treatment.

These biomarkers call on molecular biology and biochemistry. The hypothetico-deductive approach has reduced personalised medicine to several biomarkers, the interest of which has been fixed a priori and which has not made it possible to exhaust questions of early diagnosis or the theranostic approach. Thus, the search for THE biomarker making it possible to predict THE response to a biological treatment in chronic inflammatory diseases, and thus in RA, is an illusion. The multiplicity of genetic or biochemical biomarkers associated with the good clinical response or non-response to a biological treatment makes the task difficult.

Genomics, transcriptomics, epigenetics and proteomics are complementary and non-redundant pillars in this perspective.

A predictive approach of personalised or stratified medicine type is very novel in the field of chronic inflammatory rheumatisms and could make it possible to prescribe the right treatment to the right patient at the right moment, to limit the progression of the handicap by guiding the patient as quickly as possible to the treatment to which he has the greatest chance of responding, and to avoid prescribing treatments which, conversely, are associated with a low probability of response.

Adalimumab is a fully human monoclonal antibody, neutralising in a specific manner TNFα (Tumour Necrosis Factor alpha). By combining with this pro-inflammatory cytokine, Adalimumab prevents its interaction with its receptor, and thus modulates the dependent TN Fa inflammatory processes.

Some studies have focused on highlighting biomarkers making it possible to predict the response to ADA treatment in patients with RA. These studies mainly concern the characterisation of biomarkers of DNA and RNA (Krintel et al., 2012), DNA and RNA are subject to potential modifications (epigenetic, regulation of gene expression, splicing) linked to the environment before being translated into proteins which are the final effectors. The proteomic approach thus makes it possible to minimise possible variations between the expression level of biomarkers and the clinical results observed. However, these studies focus on completely biotherapy naive AR patients or an undifferentiated population of naive patients in rotation, that is to say who have had an insufficient or inadequate response to at least one biotherapy. The distinction between so-called naive populations and those having already received one or more biotherapies is very important because numerous studies have demonstrated a change in the proteome after treatment with biotherapies such as anti-TNF (Takeuchi et al., 2007) or others (Abatacept (Charles-Schoeman et al., 2018), Tociluzimab (Gabay et al., 2016)). Thus, biomarkers predictive of the response in biotherapy naive patients are capable of being modified and to be no longer relevant in patients having already received one or more biotherapies. Thus, the distinction of the therapeutic situation of the patient (naive or in rotation) is an essential criterion in the choice of predictive biomarkers. Until now, studies on ADA focusing on patients in rotation situation have mainly concentrated on the measurement of the effectiveness after administration of this molecule, in comparison with other bDMARDs (Harrold et al., 2015) and not on the characterisation of biomarkers that are specific and predictive of response to ADA.

There thus exists a need to identify novel methods and/or biomarkers making it possible to guide the practitioner in a personalised manner towards the treatment that is the most promising in terms of effectiveness for a given patient with a chronic inflammatory disease, in particular for patients suffering from rheumatoid arthritis, and notably for those who are in a situation of inadequate response to one or more biotherapy treatment(s).

The present invention responds to this technical problem vis-à-vis the response to a treatment with an anti-TNFα agent for a patient with rheumatoid arthritis not having had a sufficient therapeutic response to one or more prior biotherapy treatment(s); the inventors having identified a set of biological biomarkers of which the expression level detected in a biological sample from such a patient makes it possible to estimate the effectiveness of this treatment in this patient.

### SUMMARY OF THE INVENTION

The present invention relates to a method for estimating the effectiveness of treatment with an anti-TNFα agent in a patient with rheumatoid arthritis and not having had an adequate therapeutic response to one or more prior biotherapy treatment(s), said method comprising:

a) the in vitro measurement of the expression level of at least two biomarkers chosen from the group consisting of Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP), in a biological sample from said patient,

b) the estimation of said effectiveness of treatment with said anti-TNFα agent in said patient as a function of each expression level measured for a biomarker chosen from said group.

In particular, the method for estimating the effectiveness of treatment with an anti-TNFα agent according to the invention comprises:

a) the in vitro measurement of the expression level of at least two biomarkers chosen from the group consisting of: Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP), in a biological sample from said patient,

b1) the comparison of the expression level measured at step a) compared to that measured in a plurality of samples of patients with rheumatoid arthritis and having received a treatment with said anti-TNFα agent for which the effectiveness of treatment is known; said comparison being carried out by means of a statistical learning model using as input data the expression levels of at least two of the biomarkers measured at step a),

b2) the estimation of said effectiveness of treatment with said anti-TNFα agent in said patient as a function of the results determined by the model defined at step b1).

The set of biomarkers identified by the inventors is thus particularly suited to estimating the effectiveness of treatment with an anti-TNFα agent in a patient with rheumatoid arthritis.

The present invention furthermore relates to a system for estimating the effectiveness of treatment with an anti-TNFα agent in a patient with rheumatoid arthritis and who has had an inadequate response to at least one prior biotherapy treatment, said system comprising:
means for measuring or receiving measurement data of the expression level of at least two biomarkers chosen from a group consisting of: Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP), in a biological sample from said patient,
means for processing measurement data configured to estimate said effectiveness of treatment in said patient as a function of each expression level measured for a biomarker chosen from this group.

Preferably, the estimation method and the estimation system according to the invention make it possible to estimate the response to an anti-TNFα agent, in particular by preventing/blocking or inhibiting, directly or indirectly, the interaction between TNF alpha and its receptor. Advantageously, the estimation method and the estimation system according to the invention make it possible to estimate the response to an agent which attaches itself to TNF alpha, and in particular to estimate the response to Adalimumab.

### BRIEF DESCRIPTION OF THE FIGURES

FIG. 1 represents the ROC (Receiver Operating Characteristic) curve obtained during the evaluation of the performances of the method according to the invention with the study of the 3 biomarkers A1AT, B2M and SeleP for the prediction of the response to ADA. It represents an example of the sensitivity of the test (Y-axis) as a function of the complementary of the specificity of the test: 1—specificity (X-axis).

FIG. 2 represents the ROC (Receiver Operating Characteristic) curve obtained during evaluation of the performances of the method according to the invention with the study of the 3 variables A1AT, B2M and SeleP for the prediction of remission further to ADA. It represents an example of the sensitivity of the test (Y-axis) as a function of the complementary of the specificity of the test: 1—specificity (X-axis).

### DETAILED DESCRIPTION OF THE INVENTION

The problem encountered in the field of the invention for the development of a robust predictive test firstly consists in identifying the biomarkers which, taken together, make it possible to obtain a relevant prediction with both high specificity and high sensitivity.

That is why, according to a first aspect, the present invention relates to a method for estimating the effectiveness of treatment with an anti-TN Fa agent in a patient with rheumatoid arthritis and who has had an inadequate response to at least one prior biotherapy treatment, said method comprising, or even consisting in:

a) the in vitro measurement of the expression level of at least two biomarkers chosen from the group consisting of Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP), in a biological sample from said patient,

b) the estimation of said effectiveness of treatment with said anti-TNFα agent in said patient as a function of each expression level measured for a biomarker chosen from said group.

The inventors have in fact identified sets or combinations of relevant biomarkers for estimating the effectiveness of treatment with an anti-TNFα agent in a patient with rheumatoid arthritis and who has had an inadequate response to at least one prior biotherapy treatment, namely at least two biomarkers chosen from the group consisting of Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP).

In particular, the method for estimating the effectiveness of treatment with an anti-TNFα agent according to the invention comprises:

a) the in vitro measurement of the expression level of at least two biomarkers chosen from the group consisting of: Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP), in a biological sample from said patient,

b1) the comparison of the expression level measured at step a) compared to that measured in a plurality of samples of patients with rheumatoid arthritis and having received a treatment with said anti-TNFα agent for which the effectiveness of treatment is known; said comparison being carried out by means of a statistical learning model using as input data the expression levels of at least two of the biomarkers measured at step a),

b2) the estimation of said effectiveness of treatment with said anti-TNFα agent in said patient as a function of the results determined by the model defined at step b1).

The measurement of the expression level of the particular combinations of these particular biomarkers and their analysis notably by means of a statistical learning model make it possible to obtain a relevant estimation of the prediction of response to an anti-TNFα agent for a patient with rheumatoid arthritis.

The present invention thus relates to a personalised method for predicting the response to an anti-TNFα agent, in particular to Adalimumab of a given patient, with rheumatoid arthritis and who has had an inadequate response to at least one prior biotherapy treatment. It makes it possible to determine in a patient with rheumatoid arthritis and who has had an inadequate response to at least one prior biotherapy treatment, a level of effectiveness based on a probability of good response and of non-response to this anti-TN Fa agent or of remission and non-remission further to the treatment with this anti-TNFα agent. The method according to the invention thus makes it possible to identify responder patients and in particular patients that do not respond to the anti-TNFα agent in question.

As regards step a) of the method according to the invention, the relevant biomarkers identified by the inventors are defined hereafter.

Alpha-1-antitrypsin (A1AT) (Kim et al., 2018b), a glycoprotein synthesised by the liver, is present in most biological fluids and in stools. It belongs to the family of serpins (serine protease inhibitors) and is to a large extent responsible for the inhibiting power of blood serum vis-à-vis numerous proteases such as elastase, chymotrypsin, trypsin, cathepsin. The synthesis of alpha-1-antitrypsin increases during any inflammatory process. In 1976, Cox et al. suggested that a reduction in the concentration of A1AT contributes to the development of RA and notably to tissue destruction (Cox and Huber, 1976). A decrease in A1AT has also been observed in AR patients (Cylwik et al., 2010). A study on mice has shown that gene or protein therapy of human A1AT significantly delays the development of arthritis in an arthritic mouse model induced by collagen. Non-immune complexes between IgA and alpha-1-antitrypsin (IgA-AT) have been detected at low levels in the serum of healthy volunteers but have been found at raised levels in the serum of patients with rheumatoid arthritis and is correlated with the radiological progression of the disease in cases of early onset RA. The specific presence of citrulline form A1AT has been described in the serum of patients with RA (Chang et al., 2013). To the knowledge of the inventors, the predictive aspect of A1AT as biomarker in the treatment of RA by bDMARDs has not been reported in the literature.

Beta 2 microglobulin (B2M) (Li et al., 2016) is a small protein that forms part of the HLA (light chain) complex on the surface of nucleated cells but can also exist in free form in most biological fluids, including serum, urine and synovial fluid. This polypeptide plays an important role in the immune defences and may be used to evaluate the renal function. A plasma, serum or urinary rise in B2M has been described in AR patients. A significant decrease in the level of B2M in AR patients treated for 6 months with Auranofin (Crisp et al., 1983) has been reported. A decrease in B2M is observed in AR patients treated for osteoporosis by Alendronate compared to the placebo (Cantatore et al., 1999). However, to the knowledge of the inventors, no recent study describes the role of B2M in RA and its predictive potential in the response to bMARD treatments.

Serum Amyloid proteins A1 (SAA1) and A2 (SAA2) (Xu et al., 2005) belong to a family of proteins affiliated with the acute phase of inflammation called Serum Amyloid A (SAA). Expressed at a basal level, they are synthesised during the acute phase, at levels of 100 to 1 000 times their normal value, mainly produced by the liver under the action of pro-inflammatory cytokines. 2 classes thereof exist: i) the acute phase (A-SAA: SAA1, SAA2 and SAA3) and ii) the constitutive phase (C-SAA: SAA4). Thus, hepatic synthesis of A-SAA is strongly increased in response to inflammatory cytokines (IL-1, IL-6 and TNFα), whereas C-SAA are expressed in a constitutive manner in the absence of inflammation. The genes SAA1 and SAA2 are regulated in hepatic cells by pro-inflammatory cytokines. It has been suggested that the levels of SAA better reflect the activity of the disease in inflammatory joint diseases than the sedimentation rate and CRP traditionally used to estimate the score of the activity of the disease in RA. One study shows that the concentration of SAA remains high during treatment with DMARD unlike conventional indicators of RA (CRP and sedimentation rate) and that it could thus be a more sensitive biomarker to determine the activity of the disease. A correlation between the basal level of A-SAA and the activity of RA has been described with a decrease in the level of A-SAA one year after treatment with a bDMARD (Adalimumab, Infliximab, Etanercept or Anakinra (Connolly et al., 2012); Etanercept (Hwang et al., 2016); Golimumab (Visvanathan et al., 2009). These studies highlight the relationship between the decrease in the SAA level in the course of treatment and the associated clinical response but do not focus on the predictive value of the SAA basal level itself.

Selenoprotein P (SeleP) (Burk and Hill, 2009) is an extracellular glycoprotein, the role of which is to transport and to deliver selenium, which is an essential trace element, into the different tissues of the body. Other functions have been described notably in parasitic infection, in spermatogenesis, as nutrient status marker or instead as antioxidant. Very few studies directly link Selenoprotein P with rheumatoid arthritis. To the knowledge of the inventors, no study describes the role of SeleP in RA and its predictive potential in the response to bMARD treatments.

Lipopolysaccharide Binding Protein (LBP) is an acute phase protein which binds to various LPS molecules and to lipid A (Schumann et al., 1990). LBP is constitutively produced by hepatocytes in the liver. It binds to LPS (lipopolysaccharide) and presents it to the CD14 receptors present on monocyte cells. Thus, the main function of LBP is to improve the capacity of the host to detect LPS at the start of infection. In normal serum, LBP is constitutively present and its concentration can increase by 10 times in acute phase response (Prucha et al., 2003). Few studies recount the relationship between LBP and rheumatoid arthritis. Two studies suggest however that LBP is an inflammation marker in AR patients, its expression is lower in AR patients vs. healthy subjects and may represent a new marker of the activity of the disease in RA. A lowering in the expression of LBP has been observed during treatment with ADA and Abatacept (Charles-Schoeman et al., 2018). Using a proteomic approach, Kim et al. (Kim et al., 2018a) have recently shown that the expression of LBP was correlated with Rheumatoid Factor (RF) levels and could serve as a diagnostic marker complementary to RF of autoimmune diseases such as RA. To the knowledge of the inventors, the predictive aspect of LBP as biomarker in the treatment of RA by bDMARDs has not been reported in the literature.

Apolipoprotein C-III (or apo C-III, or apo-C3) (Norata et al., 2015) is a lipoprotein involved in the metabolism of triglycerides. The physiological functions of ApoC-III comprise the inhibition of lipoprotein lipase and hepatic lipase. ApoC-III thus represents an important lipolysis regulator. Consequently, high levels of ApoC-III have been detected in patients presenting hypertriglyceridemia. Through a proteomic approach, Blaschke et al. have shown that ApoCIII seems to be a predictor of the response after 6 months to Etanercept in AR patients. Indeed, this is significantly under-expressed before initiation of the treatment in patients who present a sufficient response (Blaschke et al., 2015). However, this study does not use a combination of biomarkers to construct a predictive model.

The complement system (Ricklin and Lambris, 2013) is one of the defence mechanisms that intervenes in the destruction of infectious agents, in the elimination of immune complexes, but also in the control of inflammatory responses and the modulation of specific immune responses. It is an activation cascade and the fraction C3 plays a central role in the activation of the complement since all of the activation routes of the complement end up in its cleavage by specific proteolytic systems into C3a and C3b. A link between the complement system and RA has been highlighted. The presence of C3 has been found in the synovial fluid of AR patients. After 12 months of treatment with Tocilizumab, a lowering of the C3 serum in correlation with the activity score of the disease has been observed in RA patients (Romano et al., 2018). Another study reports a higher expression of C3 in AR patients compared to healthy subjects and a drop in C3 specifically in responders after 12 months with Rituximab (Conigliaro et al., 2016).

Platelet Factor 4 (PF4, also called CXCL4 for chemokine (C-X-C motif) ligand 4) is a chemokine released by activated platelets and binds with strong affinity to heparin. Its major physiological role seems to be the neutralisation of molecules of heparin type on the endothelial surface of blood vessels, thus inhibiting the local activity of Antithrombin III and favouring coagulation. As strong chemical-attractant for neutrophiles and fibroblasts, PF4 probably has a role in inflammation and healing. In RA, several studies have reported an increase in PF4 in the synovial fluid and a rise in plasma levels in patients presenting cutaneous vascularity. An increase in the mRNA of PF4 has been observed in the early phase of RA (Yeo et al., 2016). Immunological complexes containing PF4 have been found specifically in the serum of AR patients suggesting a diagnostic use for RA (Ohyama et al., 2011). PF4 has been characterised as response biomarker in the treatment of rheumatoid arthritis with Infliximab (Trocmé et al., 2009) and with anti-TNFα (Etanercept, Infliximab and Adalimumab) (Nguyen et al., 2018) in biotherapy naive patients. However, to the knowledge of the inventors, the predictive aspect of PF4 as biomarker in the treatment of RA specifically in patients not having had an adequate therapeutic response to one or more prior biotherapy treatment(s) has not been reported in the literature.

COMP (cartilage oligomeric matrix protein), also designated by the name Thrombospondin 5 (TSP 5), is a glycoprotein, a member of the family of extracellular thrombospondins. This calcium binding protein is mainly present in the joint, nasal and tracheal cartilage and, to a lesser extent, in the ligaments, the meniscus and the normal tendons. It is considered as a marker of the degradation of serum cartilage in joint diseases and in particular in RA. The serum level of COMP could be considered as a potential biomarker for differentiating patients with RA and healthy individuals, with a specificity comparable to the standard biomarkers generally used to estimate the activity of the disease, such as CRP. COMP, and more specifically the specific monoclonal antibodies of COMP, have been involved in the induction of arthritis in naive mice. COMP levels were also correlated with the Larsen joint damage score at the start of RA. In addition, the levels of serum and COMP synovial fluid in patients with RA reflect not only the degradation of the cartilage, but also the Sedimentation Rate (SR) and CRP, which are indicators of the acute phase. A higher level of COMP has also been linked to the aggressiveness of the disease in RA. Similarly, a significant positive correlation has been found between the levels of COMP and the severity of the disease in early and late onset RA. After treatment with Infliximab and Etanercept, Crnkic et al. (Crnkic et al., 2003) reported a decrease in the serum concentration of COMP after 3 months which remained low after 6 months for both responders and non-responders. Another study observed a decrease in the serum concentrations of COMP after 6 months of treatment with Etanercept, but only in the remission group (Kawashiri et al., 2010). Patients having high serum levels of COMP were more likely to present a deterioration of their Larsen score (Skoumal et al., 2003).

Preferably, the method for estimating the effectiveness of treatment according to the invention is based on the in vitro measurement of the expression level of at least three of the aforementioned ten biomarkers, at least four, at least five, at least six, at least seven, at least eight, at least nine, or ten biomarkers of the group consisting of Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP).

The preferred embodiments of the method for estimating the effectiveness of treatment according to the invention comprises the in vitro measurement of the expression level of particular combinations of the following biomarkers among the list of aforementioned ten biomarkers:
at least A1AT and SAA1 or at least A1AT and SAA2, in a more preferred manner at least A1AT and B2M;
at least A1AT, B2M and LBP or at least A1AT, SAA2 and APOC3, or further and in a more preferred manner at least A1AT, B2M and SAA1 or at least A1AT, B2M and SeleP.

These sets make it possible to obtain the most relevant results in terms of estimation of the effectiveness of treatment.

With these combinations of the aforementioned two or three preferred biomarkers, it is also possible in particular to measure in vitro the expression level of at least one other biomarker chosen from the remaining eight or seven in the list of ten biomarkers described in the present description, namely Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP).

The method according to the invention makes it possible to estimate the effectiveness of treatment with an anti-TNFα agent in a patient with rheumatoid arthritis who has had an inadequate response to at least one prior biotherapy treatment.

“Patient with rheumatoid arthritis who has had an inadequate response” is taken to mean a patient with rheumatoid arthritis who has had an insufficient response to at least one prior biotherapy treatment, but also a patient with rheumatoid arthritis who has had a satisfactory response to at least one prior biotherapy treatment but who has presented at least one adverse event of moderate or severe intensity during prior treatment(s) necessitating the stoppage of treatment.

“Insufficient response to at least one prior biotherapy treatment” is taken to designate a patient not having presented a positive therapeutic response to one or more prior treatment(s) with a bDMARD (biological Disease-Modifying Anti-Rheumatic Drug). Within the scope of the treatment of chronic inflammatory diseases, current therapeutic strategies are conducted to reduce the activity of the rheumatism and the response to the treatment is assessed during the first year, generally after 6 months. Concerning rheumatoid arthritis, the response to treatment is determined by the evolution of the activity of the rheumatism according to the EULAR (European League Against Rheumatism) response. The EULAR response takes into account the activity of the rheumatism which is evaluated by the DAS28 (Disease Activity Score 28) as well at its variation. The DAS is a composite score calculated on the basis of the number of painful joints out of 28 joints, VAS (Visual Analogue Scale), and a biological inflammatory parameter: SR (sedimentation rate) or CRP (Prevoo et al., 1995). The EULAR response at a time T is defined as a function of the DAS28 score at time T and the difference between the DAS28 at time T and the initial DAS28, that is to say before treatment. Within the scope of the present invention, “insufficient response to a treatment” is taken to mean in particular a EULAR response with a DAS28 at time T greater than 3.2 or a variation in DAS28 between time T and the DAS28 before treatment less than or equal to 1.2.

Conversely, “sufficient response to a treatment” is taken to mean a EULAR response with a DAS28 at time T less than or equal to 3.2 associated with a variation in DAS28 between time T and before treatment greater than 1.2.

“Remission” at time T is taken to designate a patient presenting a DAS28 below 2.6 at time T.

Biotherapy is taken to mean a therapy resorting to the use of a bDMARD. DMARDs are a category of drugs defined by their use in rheumatoid arthritis to slow down the progression of the disease. Several types of DMARDs exist, classed in the following manner:
synthetic DMARDs (sDMARDs) which comprise conventional synthetics (csDMARDs) and targeted synthetics (tsDMARDs). csDMARDs are traditional drugs such as methotrexate, sulfasalazine, leflunomide, hydroxychloroquine, gold salts, etc. tsDMARDs are drugs which have been developed to target a particular molecular structure.
biological DMARDs (bDMARDs) which comprise original biological DMARDs (boDMARDs) and biosimilar DMARDs (bsDMARDs). bsDMARDs are those which have the same primary, secondary and tertiary structure as the original biological treatment (boDMARD) and have an effectiveness and safety similar to those of the original protein.

“Adverse event”, or AE, is taken to mean any untoward medical occurrence in a patient, whether this occurrence is linked or not to the biotherapy treatment. If this adverse event is considered by the physician as having a reasonable causality link with the procedure, the method, the act or the treatment, it is qualified as adverse effect. The expression “scientifically reasonable causality link” signifies that there exists proof or an argument making it possible to suggest, in scientific terms, a cause and effect relationship between the untoward and adverse reaction and the procedure, the method, the act or the treatment.

The intensity of adverse events is evaluated by the physician using the following classification, well-known in the field:
grade 1 mild intensity: adverse event generally transitional and not interfering with everyday activities
grade 2 moderate intensity: adverse event sufficiently discomforting to interfere with normal everyday activities
grade 3 severe intensity: adverse event considerably modifying the normal course of activities of the subject, or invalidating, or constituting a threat to the life of the subject.

The grades of all known adverse events as a function of pathologies are listed by the National Cancer Institute and accessible on the web site of the National Institutes of Health (Common Terminology Criteria for Adverse Events (CTCAE); https://safetyprofiler-ctep.nci.nih.gov/CTC/CTC.aspx). The different adverse events linked to a biotherapy treatment are notably classed in the Summary of Product Characteristics (Sm PC).

Preferably, the method according to the invention makes it possible to estimate the effectiveness of treatment with an anti-TNFα agent which is Adalimumab in a patient with rheumatoid arthritis and who has had an inadequate response to at least one prior treatment with an anti-TNFα biotherapy. Even more preferably, the method according to the invention makes it possible to estimate the effectiveness of treatment with an anti-TNFα agent in a patient with rheumatoid arthritis who has had an inadequate response to at least one prior treatment chosen from Etanercept, Abatacept, Infliximab, Tocilizumab, Rituximab, Certolizumab and Golimumab, and preferably only one of these treatments.

According to the invention, the method makes it possible to estimate the effectiveness of treatment with an anti-TNFα agent. Such an agent may be defined as being an agent that is capable of blocking, or even inhibiting, directly or indirectly, the action of TNF alpha, in particular by preventing or blocking or inhibiting, directly or indirectly, the interaction between TNF alpha and its receptor. Among these agents, the bDMARD Adalimumab may notably be cited.

In a particularly advantageous manner, the method according to the invention makes it possible to estimate the effectiveness of treatment with an anti-TNFα agent which is Adalimumab.

Any biological sample constituted of a biological fluid may be used within the scope of the invention for measuring in vitro the expression level of the aforementioned biomarkers and combinations of biomarkers, and among which may notably be cited synovial fluid, serum, plasma, saliva, urine, etc., preferably serum.

According to a preferred embodiment, the expression level of the aforementioned biomarkers and combinations of biomarkers is measured in vitro on a sample of serum from the patient for whom it is sought to estimate the effectiveness of treatment with an anti-TNFα agent which is Adalimumab.

Advantageously, the method for estimating the effectiveness of treatment according to the invention comprises at step a) the in vitro measurement of the expression level of the biomarkers or combinations of protein biomarkers.

Particularly preferred embodiments of the method according to the invention are the following, each being to apply to the combinations of biomarkers defined previously, namely at least two biomarkers chosen from the group consisting of: Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP):
The estimation of the effectiveness of treatment with Adalimumab, in a patient with rheumatoid arthritis;
The estimation of the effectiveness of treatment with Adalimumab, in a patient with rheumatoid arthritis comprising the measurement of the protein expression level of at least two markers chosen from the ten mentioned in the present description;
The estimation of the effectiveness of treatment with Adalimumab, in a patient with rheumatoid arthritis and who has had an inadequate response to at least one prior treatment with a biotherapy chosen from Etanercept, Infliximab, Tocilizumab, Abatacept, Rituximab, Certolizumab and Golimumab, preferably to only one of these treatments;
The estimation of the effectiveness of treatment with Adalimumab, in a patient with rheumatoid arthritis and who has had an inadequate response to at least one prior treatment with a biotherapy chosen from Etanercept, Tocilizumab, Infliximab, Abatacept, Rituximab, Certolizumab and Golimumab, comprising the measurement of the level of protein expression of at least two markers chosen from the ten mentioned in the present description, preferably to only one of these treatments;
The estimation of the effectiveness of treatment with Adalimumab, in a patient with rheumatoid arthritis and who has had an insufficient response to at least one prior treatment with a biotherapy chosen from Etanercept, Infliximab, Tocilizumab, Abatacept, Rituximab, Certolizumab and Golimumab, preferably to only one of these treatments;
The estimation of the effectiveness of treatment with Adalimumab, in a patient with rheumatoid arthritis and who has had an insufficient response to at least one prior treatment with a biotherapy chosen from Etanercept, Tocilizumab, Infliximab, Abatacept, Rituximab, Certolizumab and Golimumab, comprising the measurement of the level of protein expression of at least two markers chosen from the ten mentioned in the present description, preferably to only one of these treatments.

At step b1) of the estimation method according to the invention, the expression level of the biomarkers or combinations of biomarkers measured at step a) described above is compared with that measured in a plurality of samples of patients with rheumatoid arthritis and having received a treatment with an anti-TNFα agent for which the effectiveness of treatment is known.

This comparison is carried out by means of a statistical learning model using as input data the expression levels of at least two of the biomarkers measured at step a). To do so, any statistical learning model may be used, and notably the models obtained by logistic regression methods, discriminant analysis, neural networks, decision tree learning, support vector machines (SVM), or aggregation of models.

Preferably, in the method for estimating the effectiveness of treatment with an anti-TNFα agent according to the invention, the expression levels of each biomarker measured at step a) are used to obtain a score linked to the estimation of the effectiveness of this treatment in said patient, said score being compared with at least one predetermined threshold so as to classify the prognosis among a plurality of classes. In this embodiment, it is notably possible to use a plurality of classes which comprises at least two classes of which one class of non-response to the treatment with said anti-TNFα agent. The two classes may for example be derived from so-called “non-responder” patients, who present an insufficient response to the treatment and so-called “responder” patients who present a sufficient response to the treatment. Still in this embodiment, the estimation of the effectiveness of treatment in the patient with rheumatoid arthritis and who has had an inadequate response to at least one prior biotherapy treatment comprises the comparison of said score with a predetermined threshold below which poor effectiveness is predicted and above which good effectiveness is predicted.

Preferably, the method for estimating the effectiveness of treatment with an anti-TNFα agent according to the invention uses at step b1) a learning model based on a prior analysis of samples of a cohort comprising patients treated with said anti-TNFα agent presenting good responses to the treatment and patients treated with said anti-TNFα agent presenting poor responses to the treatment. In this embodiment, the learning model is preferably based on a prior analysis which comprises the application of a method for learning and selecting variables. Advantageously, logistic regression will be used as method for learning and selecting variables.

Still in this embodiment based on a prior analysis which comprises the application of a method for learning and selecting variables, the expression levels of the biomarkers or combinations of biomarkers measured at step a) are weighted as a function of the prior analysis of the cohort comprising patients treated with said anti-TNFα agent presenting good responses to the treatment and patients treated with said anti-TNFα agent presenting poor responses to the treatment to deliver the score linked to the estimation of the effectiveness of treatment.

Still in this embodiment based on a prior analysis which comprises the application of a method for learning and selecting variables, the method for estimating the effectiveness of treatment according to the invention may use a method of learning by decision tree. According to this embodiment, the expression levels of the biomarkers or combinations of biomarkers measured at step a) are compared with a reference value at each node of the tree.

The reference values may be obtained by the analysis of the expression level of the biomarkers or combinations of biomarkers in biological samples of a set of patients with rheumatoid arthritis before treatment so as to have available a set of data on the expression levels of the biomarkers associated with each biological sample of each patient.

These reference values can change over time as a function of the results obtained with other patients that complete the number of results serving to define the threshold value.

According to a second aspect, the invention also relates to a system for estimating the effectiveness of treatment with an anti-TNFα agent in a patient with rheumatoid arthritis and who has had an inadequate response to at least one prior biotherapy treatment, said system comprising:
means for measuring or receiving measurement data of the expression level of at least two biomarkers chosen from a group consisting of: Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4) and Cartilage Oligomeric Matrix Protein (COMP), in a biological sample from said patient,
means for processing measurement data configured to estimate said effectiveness of treatment in said patient as a function of each expression level measured for a biomarker chosen from this group.

Among the means for measuring the expression level of the biomarkers or combinations of biomarkers selected, it is notably possible to cite specific reagents of each of the biomarkers such as enzymes, substrates or antibodies which may be used in methods, such as among others nephelometry, chemoluminescence, immunoturbidimetry, flow cytometry, ELISA, etc, but also physical means such as mass spectrometry analysis methods for example.

The system according to the invention may moreover contain means for receiving measurement data, thus making it possible to estimate, for said patient, the effectiveness of response to the treatment with the anti-TNFα agent, from data supplied for example by a practitioner having had the expression level of biological biomarkers measured such as described previously.

The reception means may notably comprise transmission/reception means for exchanging with a remote server through a communication network such as an intranet network or the secure internet network. The device may also comprise input means such as a keyboard.

The data processing means may notably call on database management, code instructions, the development of software comprising an algorithmic brick, an interface to enable the user to consult the results, etc. These different elements may be recorded on a storage support such as a hard disc, a CD ROM, a USB key, or any other storage support known to those skilled in the art.

They may be implemented by a device which may be fixed or mobile. The device is for example a personal computer, a mobile telephone, an electronic tablet, or any other type of terminal known to those skilled in the art.

In an alternative, the system may also comprise transmission means for transmitting, still via intranet or internet for example, the results of the estimation of the effectiveness of treatment in the patient concerned.

According to another advantageous alternative, the system according to the invention comprises means for receiving the effectiveness data obtained, and does so in order to complete and enrich the reference values in view of the result of treatment obtained with regard to the expression level of the selected biomarkers.

### Examples

### Materials & Methods

### Development of the Predictive Model

The link between the response to the biotherapy, or the remission, and each variable is analysed through a logistic regression model on a set of data. The variable to explain is in the first case the good response during the first year of treatment, and in the second case the remission during the first year of treatment.

Firstly, a pre-selection of the variables to include in the multivariate model is carried out. To do so, the predictive capacity of each explanatory variable is analysed individually. The biomarkers are analysed in a quantitative and qualitative manner. A method for selecting variables is put in place to conserve uniquely the relevant variables that will next be introduced into a multivariate model. The biomarkers are preselected if they have:
In quantitative form, a p-value <0.20 or an AUC>0.60
In qualitative form, a p-value <0.05 and an AUC>0.65

The criteria chosen are voluntarily wide to include significant variables, but also variables presenting trends in the multivariate model.

The preselected biomarkers exhibit relevant trends to analyse. Multivariate models with the different possible combinations of these biomarkers are constructed, and the AUC calculated. Models having an AUC>0.70 are considered as relevant and are conserved.

The models thereby constructed make it possible to weight the dosage results of each of the specific biomarkers to obtain a probability of response or remission. The coefficients of each model make it possible to calculate from the dosage values of each patient an associated probability of response, or remission. The performance characteristics (AUC (area under curve), sensitivity and specificity, PPV (positive predictive value) and NPV (negative predictive value)) of each model are calculated to define its relevance.

An AUC>0.70 is considered as an acceptable discrimination, an AUC>0.80 demonstrates very good discrimination capacity. A level of probability threshold is fixed to calculate specificity and sensitivity. This optimal threshold is determined on the basis of the Youden index. At this threshold, patients may be classified as a function of the following table 1:

TABLE 1

| Responder | Non-responder | |
|---|---|---|
| Positive test (prob > threshold) | TP | FP |
| Negative test (prob < threshold) | FN | TN |

TP (true positives) represents the number of responder individuals with a positive test,
FP (false positives) represents the number of non-responder individuals with a positive test,
FN (false negatives) represents the number of responder individuals with a negative test,
TN (true negatives) represents the number of non-responder individuals with a negative test.

The sensitivity, or the probability that the test is positive if the patient is a responder, is measured in sufferers only. It is given by:

$$\frac{T P}{T P + F N}$$

The specificity is measured in non-sufferers only. The specificity, or the probability of obtaining a negative test in non-responders is given by:

$$\frac{T N}{T N + F P}$$

The sensitivity of the test measures its capacity to provide a positive result when the patient is a responder. The specificity measures the capacity of the test to give a negative result when the patient is a non-responder.

The positive predictive value (PPV) is the probability that the patient is a responder when the test is positive.

$$PPV = \frac{T P}{T P + F P}$$

The negative predictive value (NPV) is the probability that the patient is a non-responder when the test is negative.

$$NPV = \frac{T N}{T N + F N}$$

### Results:

Out of a learning cohort consisting of 54 patients with RA, the models constructed have the characteristics presented in table 2 below which shows the combinations of 2 or 3 biomarkers with AUC>0.75. All the combinations including at least 2 of the 10 biomarkers among A1AT, B2M, SAA2, SeleP, LBP, C3, APOC3, SAA1, PF4, COMP provide relevant results.

TABLE 2

| | | | | | | | | | | | AUC | AUC |
|---|---|---|---|---|---|---|---|---|---|---|---|---|
| N | A1AT | B2M | SAA2 | SeleP | LMP | C3 | APOC3 | SAA1 | PF4 | COMP | response | remission |
| 3 | X | X | | | | | | X | | | 0.86 | 0.87 |
| | X | X | | X | | | | | | | 0.85 | 0.87 |
| | X | X | | | X | | | | | | 0.83 | 0.89 |
| | X | | X | | | | X | | | | 0.84 | 0.87 |
| | X | | X | | | | | | | X | 0.87 | 0.82 |
| | X | | | | | | | X | X | | 0.87 | 0.82 |
| | X | | X | | | | | X | | | 0.85 | 0.84 |
| | X | X | X | | | | | | | | 0.84 | 0.85 |
| | X | | X | X | | | | | | | 0.84 | 0.85 |
| | X | X | | | | | | | | X | 0.83 | 0.86 |
| | X | | | X | | | | X | | | 0.81 | 0.88 |
| | X | X | | | | | X | | | | 0.81 | 0.87 |
| | | X | X | | | | | | | X | 0.84 | 0.84 |
| | X | | X | | X | | | | | | 0.84 | 0.84 |
| | X | X | | | | | | | X | | 0.83 | 0.85 |
| | X | X | | | | X | | | | | 0.83 | 0.84 |
| | | X | | X | | | | X | | | 0.82 | 0.85 |
| | | X | | | | | | | X | X | 0.8 | 0.86 |
| | X | | | | | | X | X | | | 0.82 | 0.84 |
| | X | | | X | | | | | X | | 0.82 | 0.84 |
| | X | | | | X | | | X | | | 0.82 | 0.84 |
| | X | | | | | | | X | | X | 0.86 | 0.79 |
| | | X | | | X | | | | | X | 0.78 | 0.87 |
| | | X | | X | | | | | | X | 0.77 | 0.88 |
| | X | | X | | | X | | | | | 0.84 | 0.8 |
| | | | X | X | | | | | | X | 0.81 | 0.83 |
| | | X | | | | | | X | | X | 0.8 | 0.84 |
| | | X | | | X | | | | X | | 0.78 | 0.86 |
| | | X | | | X | | | X | | | 0.77 | 0.87 |
| | X | | | X | | | | | | X | 0.82 | 0.82 |
| | X | | | | | X | | | X | | 0.84 | 0.79 |
| | X | | | | X | | | | X | | 0.82 | 0.81 |
| | X | | | | | X | | X | | | 0.82 | 0.81 |
| | X | | X | | | | | | X | | 0.82 | 0.81 |
| | X | | | | | | X | | | X | 0.82 | 0.81 |
| | | X | | | | X | | | X | | 0.81 | 0.82 |
| | | X | X | X | | | | | | | 0.79 | 0.84 |
| | | X | | X | | | | | X | | 0.77 | 0.86 |
| | X | | | X | | | X | | | | 0.76 | 0.87 |
| | | X | | | X | X | | | | | 0.76 | 0.87 |
| | | X | | X | X | | | | | | 0.75 | 0.88 |
| | X | | | | | | | | X | X | 0.84 | 0.78 |
| | X | | | | X | | | | | X | 0.82 | 0.8 |
| | | X | X | | | | X | | | | 0.82 | 0.8 |
| | | | X | | X | | X | | | | 0.81 | 0.81 |
| | X | | | | | | X | | X | | 0.81 | 0.81 |
| | | | X | X | | | X | | | | 0.79 | 0.83 |
| | | X | X | | X | | | | | | 0.78 | 0.84 |
| | | X | | | | X | | | | X | 0.77 | 0.85 |
| | | | X | X | X | | | | | | 0.77 | 0.85 |
| | | X | | X | | | X | | | | 0.77 | 0.85 |
| | | | X | | X | | | | | X | 0.81 | 0.8 |
| | | | X | | | X | X | | | | 0.8 | 0.81 |
| | | | X | | | | X | | | X | 0.86 | 0.75 |
| | | X | | | | | X | | | X | 0.78 | 0.83 |
| | | X | | | X | | X | | | | 0.76 | 0.85 |
| | | | | X | | | | | X | X | 0.8 | 0.8 |
| | | X | X | | | | | X | | | 0.8 | 0.8 |
| | | X | | | | | | X | X | | 0.79 | 0.81 |
| | | | X | | X | | | X | | | 0.78 | 0.82 |
| | | | X | | X | | | | X | | 0.78 | 0.82 |
| | | | | X | X | | | | | X | 0.77 | 0.83 |
| | X | | | X | X | | | | | | 0.77 | 0.83 |
| | | X | X | | | X | | | | | 0.81 | 0.78 |
| | | | | | X | | | | X | X | 0.8 | 0.79 |
| | X | | | | X | | X | | | | 0.77 | 0.82 |
| | | | | X | | | | X | X | | 0.77 | 0.82 |
| | | X | | | | | X | X | | | 0.77 | 0.82 |
| | | | | X | | | | X | | X | 0.76 | 0.83 |
| | | X | | | | X | X | | | | 0.76 | 0.83 |
| | X | | | | | X | | | | X | 0.81 | 0.77 |
| | | | | | X | | | X | | X | 0.79 | 0.79 |
| | X | | | | | X | X | | | | 0.76 | 0.82 |
| | | | | X | X | | | | X | | 0.76 | 0.82 |
| | | X | | | | X | | X | | | 0.76 | 0.82 |
| | | | | | | | | X | X | X | 0.8 | 0.77 |
| | | | X | | | | X | | X | | 0.79 | 0.78 |
| | | | X | | | | | X | | X | 0.79 | 0.78 |
| | | | X | X | | | | | X | | 0.77 | 0.8 |
| | | X | | | | | X | | X | | 0.77 | 0.8 |
| | | | | | X | | | X | X | | 0.77 | 0.8 |
| | X | | | X | | X | | | | | 0.76 | 0.81 |
| | | | | X | | X | | | X | | 0.76 | 0.81 |
| | X | | | | X | X | | | | | 0.77 | 0.79 |
| | | | X | | | | X | X | | | 0.77 | 0.79 |
| | | | | | X | X | | | | X | 0.77 | 0.79 |
| | | | | | | X | X | | | X | 0.77 | 0.79 |
| | | | X | | | X | | | | X | 0.79 | 0.76 |
| | | X | X | | | | | | X | | 0.78 | 0.77 |
| | | | | | | X | | X | X | | 0.77 | 0.78 |
| | | | | | X | | X | | | X | 0.79 | 0.75 |
| | | | | X | | | X | | | X | 0.75 | 0.79 |
| | | | | | X | X | | | X | | 0.75 | 0.79 |
| | | | X | | | | | X | X | | 0.76 | 0.75 |
| 2 | X | X | | | | | | | | | 0.8 | 0.83 |
| | X | | X | | | | | | | | 0.81 | 0.78 |
| | X | | | | | | | X | | | 0.81 | 0.78 |
| | X | | | | | | | | X | | 0.8 | 0.75 |
| | X | | | X | | | | | | | 0.76 | 0.79 |
| | X | | | | X | | | | | | 0.75 | 0.77 |

Taking for example the model with 3 variables A1AT, B2M and SeleP, the characteristics obtained are presented in table 3 below.

TABLE 3
Model with 3 variables: MAT, B2M and SeleP,

| | Response | Remission |
|---|---|---|
| AUC | 0.85 | 0.87 |
| Sensitivity | 0.90 | 0.67 |
| Specificity | 0.75 | 0.89 |
| PPV | 0.70 | 0.75 |
| NPV | 0.92 | 0.84 |

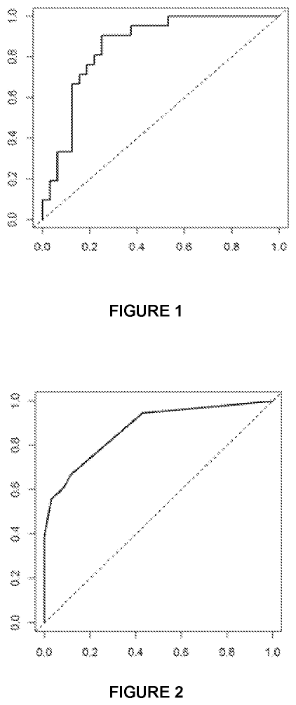

The corresponding ROC curves are represented in appended FIGS. 1 and 2.

### REFERENCES

Blaschke, S., Rinke, K., Maring, M., Flad, T., Patschan, S., Jahn, O., Mueller, C. A., Mueller, G. A., and Dihazi, H. (2015). Haptoglobin-α1, -α2, vitamin D-binding protein and apolipoprotein C-III as predictors of etanercept drug response in rheumatoid arthritis. Arthritis Res. Ther. 17.
Burk, R. F., and Hill, K. E. (2009). Selenoprotein P—Expression, Functions, and Roles in Mammals. Biochim. Biophys. Acta 1790, 1441-1447.
Cantatore, F. P., Acquista, C. A., and Pipitone, V. (1999). Evaluation of bone turnover and osteoclastic cytokines in early rheumatoid arthritis treated with alendronate. J. Rheumatol. 26, 2318-2323.
Chang, X., Zhao, Y., Wang, Y., Chen, Y., and Yan, X. (2013). Screening citrullinated proteins in synovial tissues of rheumatoid arthritis using 2-dimensional western blotting. J. Rheumatol. 40, 219-227.
Charles-Schoeman, C., Gugiu, G. B., Ge, H., Shahbazian, A., Lee, Y. Y., Wang, X., Furst, D. E., Ranganath, V. K., Maldonado, M., Lee, T., et al. (2018). Remodeling of the HDL proteome with treatment response to abatacept or Adalimumab in the AMPLE trial of patients with rheumatoid arthritis. Atherosclerosis 275, 107-114.
Conigliaro, P., Triggianese, P., Chimenti, M. S., Lucchetti, R., Kroegler, B., and Perricone, R. (2016). Serological markers associated with disease activity in patients with rheumatoid arthritis treated with rituximab. J. Int. Med. Res. 44, 53-57.
Connolly, M., Mullan, R. H., McCormick, J., Matthews, C., Sullivan, O., Kennedy, A., FitzGerald, O., Poole, A. R., Bresnihan, B., Veale, D. J., et al. (2012). Acute-phase serum amyloid A regulates tumor necrosis factor α and matrix turnover and predicts disease progression in patients with inflammatory arthritis before and after biologic therapy. Arthritis Rheum. 64, 1035-1045.
Cox, D. W., and Huber, O. (1976). Rheumatoid arthritis and alpha-1-antitrypsin. Lancet Lond. Engl. 1, 1216-1217.
Crnkic, M., Månsson, B., Larsson, L., Geborek, P., Heinegård, D., and Saxne, T. (2003). Serum cartilage oligomeric matrix protein (COMP) decreases in rheumatoid arthritis patients treated with infliximab or etanercept. Arthritis Res. Ther. 5, R181-185.
Cylwik, B., Chrostek, L., Gindzienska-Sieskiewicz, E., Sierakowski, S., and Szmitkowski, M. (2010). Relationship between serum acute-phase proteins and high disease activity in patients with rheumatoid arthritis. Adv. Med. Sci. 55, 80-85.
Gabay, C., McInnes, I. B., Kavanaugh, A., Tuckwell, K., Klearman, M., Pulley, J., and Sattar, N. (2016). Comparison of lipid and lipid-associated cardiovascular risk marker changes after treatment with tocilizumab or Adalimumab in patients with rheumatoid arthritis. Ann. Rheum. Dis. 75, 1806-1812.
Harrold, L. R., Reed, G. W., Kremer, J. M., Curtis, J. R., Solomon, D. H., Hochberg, M. C., and Greenberg, J. D. (2015). The comparative effectiveness of abatacept versus anti-tumour necrosis factor switching for rheumatoid arthritis patients previously treated with an anti-tumour necrosis factor. Ann. Rheum. Dis. 74, 430-436.
Hwang, Y. G., Balasubramani, G. K., Metes, I. D., Levesque, M. C., Bridges, S. L., and Moreland, L. W. (2016). Differential response of serum amyloid A to different therapies in early rheumatoid arthritis and its potential value as a disease activity biomarker. Arthritis Res. Ther. 18.
Kawashiri, S.-Y., Kawakami, A., Ueki, Y., Imazato, T., Iwamoto, N., Fujikawa, K., Aramaki, T., Tamai, M., Nakamura, H., Origuchi, T., et al. (2010). Decrement of serum cartilage oligomeric matrix protein (COMP) in rheumatoid arthritis (RA) patients achieving remission after 6 months of etanercept treatment: comparison with CRP, IgM-RF, MMP-3 and anti-CCP Ab. Jt. Bone Spine Rev. Rhum. 77, 418-420.
Kim, D., Mun, S., Lee, J., Park, A., Seok, A., Chun, Y.-T., and Kang, H.-G. (2018a). Proteomics analysis reveals differential pattern of widespread protein expression and novel role of histidine-rich glycoprotein and lipopolysaccharide-binding protein in rheumatoid arthritis. Int. J. Biol. Macromol. 109, 704-710.
Kim, M., Cai, Q., and Oh, Y. (2018b). Therapeutic potential of alpha-1 antitrypsin in human disease. Ann. Pediatr. Endocrinol. Metab. 23, 131-135.
Krintel, S. B., Palermo, G., Johansen, J. S., Germer, S., Essioux, L., Benayed, R., Badi, L., Ostergaard, M., and Hetland, M. L. (2012). Investigation of single nucleotide polymorphisms and biological pathways associated with response to TNFα inhibitors in patients with rheumatoid arthritis. Pharmacogenet. Genomics 22, 577-589.
Li, L., Dong, M., and Wang, X.-G. (2016). The Implication and Significance of Beta 2 Microglobulin: A Conservative Multifunctional Regulator. Chin. Med. J. (Engl.) 129, 448-455.
Nguyen, M. V. C., Baillet, A., Romand, X., Trocmé, C., Courtier, A., Marotte, H., Thomas, T., Soubrier, M., Miossec, P., Tébib, J., et al. (2018). Prealbumin, platelet factor 4 and S100A12 combination at baseline predicts good response to TNF alpha inhibitors in Rheumatoid Arthritis. Jt. Bone Spine Rev. Rhum.
Norata, G. D., Tsimikas, S., Pirillo, A., and Catapano, A. L. (2015). Apolipoprotein C-III: From Pathophysiology to Pharmacology. Trends Pharmacol. Sci. 36, 675-687.
Ohyama, K., Ueki, Y., Kawakami, A., Kishikawa, N., Tamai, M., Osaki, M., Kamihira, S., Nakashima, K., and Kuroda, N. (2011). Immune complexome analysis of serum and its application in screening for immune complex antigens in rheumatoid arthritis. Clin. Chem. 57, 905-909.
Prevoo, M. L., van 't Hof, M. A., Kuper, H. H., van Leeuwen, M. A., van of Putte, L. B., and van Riel, P. L. (1995). Modified disease activity scores that include twenty-eight-joint counts. Development and validation in a prospective longitudinal study of patients with rheumatoid arthritis. Arthritis Rheum. 38, 44-48.
Prucha, M., Herold, I., Zazula, R., Dubska, L., Dostal, M., Hildebrand, T., and Hyanek, J. (2003). Significance of lipopolysaccharide-binding protein (an acute phase protein) in monitoring critically ill patients. Crit. Care Lond. Engl. 7, R154-159.
Rendas-Baum, R., Wallenstein, G. V., Koncz, T., Kosinski, M., Yang, M., Bradley, J., and Zwillich, S. H. (2011). Evaluating the effectiveness of sequential biologic therapies for rheumatoid arthritis patients with an inadequate response to tumor necrosis factor-α inhibitors. Arthritis Res. Ther. 13, R25.
Ricklin, D., and Lambris, J. D. (2013). Complement in immune and inflammatory disorders: therapeutic interventions. J. Immunol. Baltim. Md. 1950 190, 3839-3847.
Romano, C., Del Mastro, A., Sellitto, A., Solaro, E., Esposito, S., and Cuomo, G. (2018). Tocilizumab reduces complement C3 and C4 serum levels in rheumatoid arthritis patients. Clin. Rheumatol. 37, 1695-1700.
Schumann, R. R., Leong, S. R., Flaggs, G. W., Gray, P. W., Wright, S. D., Mathison, J. C., Tobias, P. S., and Ulevitch, R. J. (1990). Structure and function of lipopolysaccharide binding protein. Science 249, 1429-1431.
Skoumal, M., Kolarz, G., and Klingler, A. (2003). Serum levels of cartilage oligomeric matrix protein. A predicting factor and a valuable parameter for disease management in rheumatoid arthritis. Scand. J. Rheumatol. 32, 156-161.
Smolen, J. S., Landewé, R., Bijlsma, J., Burmester, G., Chatzidionysiou, K., Dougados, M., Nam, J., Ramiro, S., Voshaar, M., van Vollenhoven, R., et al. (2017). EULAR recommendations for the management of rheumatoid arthritis with synthetic and biological disease-modifying antirheumatic drugs: 2016 update. Ann. Rheum. Dis. 76, 960-977.
Souto, A., Maneiro, J. R., and Gómez-Reino, J. J. (2016). Rate of discontinuation and drug survival of biologic therapies in rheumatoid arthritis: a systematic review and meta-analysis of drug registries and health care databases. Rheumatol. Oxf. Engl. 55, 523-534.
Takeuchi, T., Nakanishi, T., Tabushi, Y., Hata, A., Shoda, T., Kotani, T., Shimizu, A., Takubo, T., Makino, S., and Hanafusa, T. (2007). Serum protein profile of rheumatoid arthritis treated with anti-TNF therapy (infliximab). J. Chromatogr. B Analyt. Technol. Biomed. Life. Sci. 855, 66-70.
Trocmé, C., Marotte, H., Baillet, A., Pallot-Prades, B., Garin, J., Grange, L., Miossec, P., Tebib, J., Berger, F., Nissen, M. J., et al. (2009). Apolipoprotein A-I and platelet factor 4 are biomarkers for infliximab response in rheumatoid arthritis. Ann. Rheum. Dis. 68, 1328-1333.
Visvanathan, S., Wagner, C., Rojas, J., Kay, J., Dasgupta, B., Matteson, E. L., Mack, M., Baker, D. G., and Rahman, M. U. (2009). E-selectin, interleukin 18, serum amyloid a, and matrix metalloproteinase 9 are associated with clinical response to golimumab plus methotrexate in patients with active rheumatoid arthritis despite methotrexate therapy. J. Rheumatol. 36, 1371-1379.
Xu, Y., Yamada, T., Satoh, T., and Okuda, Y. (2005). Measurement of serum amyloid A1 (SAA1), a major isotype of acute phase SAA. Clin. Chem. Lab. Med. CCLM 44, 59-63.
Yeo, L., Adlard, N., Biehl, M., Juarez, M., Smallie, T., Snow, M., Buckley, C. D., Raza, K., Filer, A., and Scheel-Toellner, D. (2016). Expression of chemokines CXCL4 and CXCL7 by synovial macrophages defines an early stage of rheumatoid arthritis. Ann. Rheum. Dis. 75, 763-771.

1.-15. (canceled)

16. Method for estimating the effectiveness of treatment with an anti-TNFα agent in a patient with rheumatoid arthritis and who has had an inadequate response to at least one prior biotherapy treatment, the method comprising:
a) an in vitro measurement of an expression level of at least two biomarkers chosen from a group of biomarkers consisting of:
Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4), and Cartilage Oligomeric Matrix Protein (COMP), in a biological sample from the patient; and
b) an estimation of an effectiveness of treatment with said anti-TNFα agent in the patient as a function of each expression level measured for a biomarker chosen from the group of biomarkers.

17. Method for estimating the effectiveness of treatment with an anti-TNFα agent in a patient with rheumatoid arthritis and who has had an inadequate response to at least one prior biotherapy treatment, the method comprising:
a) an in vitro measurement of an expression level of at least two biomarkers chosen from a group of biomarkers consisting of:
Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4), and Cartilage Oligomeric Matrix Protein (COMP), in a biological sample from the patient;
b1) a comparison of the expression level measured at step a) compared to that measured in a plurality of samples of patients with rheumatoid arthritis and having received a treatment with said anti-TNFα agent for which the effectiveness of treatment is known; the comparison being carried out by means of a statistical learning model using as input data the expression levels of at least two of the biomarkers measured at step a); and
b2) an estimation of an effectiveness of treatment with said anti-TNFα agent in the patient as a function of the results determined by the model defined at step b1).

18. Method for estimating the effectiveness of treatment with an anti-TNFα agent according to claim 16, wherein the expression levels of each biomarker measured at step a) are used to obtain a score linked to the estimation of the effectiveness of treatment in the patient, the score being compared with at least one predetermined threshold so as to classify the prognosis among a plurality of classes.

19. Method for estimating the effectiveness of treatment with an anti-TNFα agent according to claim 18, wherein the plurality of classes comprises at least two classes of which a class of non-response to the treatment with the anti-TNFα agent.

20. Method for estimating the effectiveness of treatment with an anti-TNFα agent according to claim 18, wherein the estimation of the effectiveness of treatment in the patient comprises a comparison of the score with a predetermined threshold below which poor effectiveness is predicted and above which good effectiveness is predicted.

21. Method for estimating the effectiveness of treatment with an anti-TNFα agent according to claim 17, wherein the learning model is based on a prior analysis of a cohort comprising patients treated with said anti-TNFα agent presenting good responses to the treatment and patients treated with said anti-TNFα agent presenting poor responses to the treatment.

22. Method for estimating the effectiveness of treatment with an anti-TNFα agent according to claim 21, wherein the prior analysis comprises an application of a method for learning and selecting variables.

23. Method for estimating the effectiveness of treatment with an anti-TNFα agent according to claim 22, wherein the method for learning and selecting variables is a logistic regression.

24. Method for estimating the effectiveness of treatment with an anti-TNFα agent according to claim 21, wherein the expression levels are weighted as a function of the prior analysis of the cohort to derive a score.

25. Method for estimating the effectiveness of treatment with an anti-TNFα agent according to claim 22, wherein the method for learning comprises a decision tree wherein each node corresponds to a comparison of the expression level measured at step a) with a reference value.

26. Method for estimating the effectiveness of treatment with an anti-TNFα agent according to claim 16, wherein the agent is capable of blocking or inhibiting, directly or indirectly, the action of TNFα.

27. Method for estimating the effectiveness of treatment with an anti-TNFα agent according to claim 16, wherein the patient has had an inadequate response to at least one prior treatment chosen from Etanercept, Abatacept, Infliximab, Tocilizumab, Rituximab, Certolizumab, and Golimumab.

28. Method for estimating the effectiveness of treatment with an anti-TNFα agent according to claim 16, wherein the biological sample is constituted of a sample of biological fluid.

29. Method for estimating the effectiveness of treatment with an anti-TNFα agent according to claim 16, wherein the biomarker(s) of which the expression level is measured at step a) is a/are protein biomarker(s).

30. System for estimating the effectiveness of treatment with an anti-TNFα agent in a patient with rheumatoid arthritis and who has had an inadequate response to at least one prior biotherapy treatment, the system comprising:
means for measuring or receiving measurement data of an expression level of at least two biomarkers chosen from a group of biomarkers consisting of: Alpha1 antitrypsin (A1AT), Beta-2-microglobulin (B2M), Serum Amyloid A-2 (SAA2), Selenoprotein P (SeleP), Lipopolysaccharide-Binding Protein (LBP), Complement C3 (C3), Apolipoprotein C-III (APOC3), Serum Amyloid A-1 (SAA1), Platelet Factor 4 (PF4), and Cartilage Oligomeric Matrix Protein (COMP), in a biological sample from the patient; and
means for processing measurement data configured to estimate an effectiveness of treatment in the patient as a function of each expression level measured for a biomarker chosen from the group of biomarkers.

31. Method for estimating the effectiveness of treatment with an anti-TNFα agent according to claim 16, wherein the agent prevents or blocks or inhibits, directly or indirectly, the interaction between TNFα and its receptor.

32. Method for estimating the effectiveness of treatment with an anti-TNFα agent according to claim 16, wherein the agent is Adalimumab.

33. Method for estimating the effectiveness of treatment with an anti-TNFα agent according to claim 16, wherein the biological sample is serum.
